(19) United States
(12) Patent Application Publication
(10) Pub. No.: US 2017/0325806 A1
(43) Pub. Date: Nov. 16, 2017

(54) Suturing Device

(71) Applicant: Orthopedic Technology Innovations LLC, Bethlehem, PA (US)

(72) Inventors: Gregory Adams, Washington Crossing, PA (US); Stephen A. Brigido, Bethlehem, PA (US)

(21) Appl. No.: 15/591,876

(22) Filed: May 10, 2017

Related U.S. Application Data
(60) Provisional application No. 62/334,700, filed on May 11, 2016.

(51) Int. Cl. A61B 17/04; A61B 17/28; A61B 17/06; A61B 17/29

(52) U.S. Cl. CPC A61B 17/0469; A61B 17/282; A61B 17/0483; A61B 2017/0464; A61B 2017/0609; A61B 2017/2933; A61B 2017/0472

(57) ABSTRACT

Disclosed is a suturing device comprising a lower jaw rigidly attached to an elongated suturing device body, an upper jaw mobile with respect to the lower jaw, and an activation mechanism for actuating movement of the upper jaw, wherein each jaw comprises at least two apertures or channels within the lower jaw configured and dimensioned to allow a suture thread to pass through. Also disclosed is a suturing device comprising an elongated drive shaft, a handle assembly comprising a tubular handle body, at least one movable element connected to the drive shaft, and an activation mechanism comprising an rotatable activation knob for actuating movement of the at least one movable element. A suturing device combining the above-described jaw elements and activation mechanism is additionally provided. Further, kits comprising disclosed devices and surgical methods utilizing the same are also provided.

### FIELD OF THE INVENTION

The present invention relates generally to methods and instruments for passing a suture through tissue.

### DISCUSSION AND COMPARISON WITH RELEVANT PRIOR ART

The present invention provides a suturing device comprising: (a) a lower jaw rigidly attached to an elongated suturing device body, said lower jaw comprises a needle retaining means for securing a pair of tissue perforating needles in position, and at least two apertures or channels within the lower jaw configured and dimensioned to allow a suture thread to pass through, and (b) an upper jaw mobile with respect to the lower jaw, said upper jaw comprises a needle capturing means for capturing the pair of tissue perforating needles, and at least two apertures or channels within the upper jaw configured and dimensioned to allow a suture thread to pass through, and (c) an activation mechanism for actuating movement of the upper jaw, wherein the needle retaining means and needle capturing means are configured such that when the upper jaw closes down on the lower jaw, and subsequently moves away from the lower jaw, the pair of tissue perforating needles is released by the lower jaw and simultaneously transferred to the upper jaw.

The present invention also provides a suturing device comprising: (a) an elongated drive shaft comprising a first end and a second end housed in a protective tube, (b) a handle assembly comprising a tubular handle body, located on the first end of the drive shaft, (c) at least one movable element located on the second end of the drive shaft, which movable element is connected to the drive shaft, and (d) an activation mechanism for actuating movement of the at least one movable element, wherein: the activation mechanism comprises a rotatable actuation knob, movement of the drive shaft in its longitudinal direction is actuatable by rotation of the rotatable actuation knob about the longitudinal axis of the drive shaft, and movement of the movable element is actuated by movement of the drive shaft in its longitudinal direction. A suturing device combining the above-described jaw elements and activation mechanism for said jaw elements is additionally provided.

In use, a pair of tissue perforating needles, preferably attached to two ends of the same suture thread, is positioned loosely on the lower jaw. Said pair of tissue perforating needles is pushed through a body tissue to be sutured as the upper jaw closes down upon the lower jaw, and the suture thread is pulled through said tissue as the upper jaw re-opens away from the lower jaw. Numerous suturing devices comprising movable jaws have been proposed for use during surgical procedures requiring small-incisions or handling of tissues in difficult to access areas.

U.S. Pat. Nos. 5,454,823 and 5,690,653 issued to Richardson et al. teach a suturing apparatus comprising upper and lower jaw elements selectively movable between a first position in which portions of the respective jaw elements are positioned relatively close together and a second position in which said portions of said respective jaw elements are positioned relative further apart, each of said jaw elements being provided with a respective recess or channel arranged to receive a portion of an elongate surgical incision member (i.e., needle) or length of surgical thread, and respective selectively actuable securing means arranged to secure said surgical incision member or length of surgical thread in a respective said recess relative to said respective jaw elements. However, Richardson's design can be distinguished from that of the present invention in a number of ways.

First, each jaw in Richardson's design comprises only a single V or U shaped recess or channel for receiving a single surgical incision member, and mechanically actuated securing means for securing the incision member within said recess. In contrast, each of the upper and lower jaw of the suturing device described herein has within them at least two apertures or two channels configured and dimensioned to allow a suture thread to pass through, and no mechanical actuation is required to secure the needle. Also, unlike Richardson's design, the suturing device described herein does not require a releasing means engageable with said securing means.

In a preferred embodiment of the present invention a suture thread, having a tissue perforating needle located at each end, is preloaded onto the suturing device, with the two needles positioned in the at least two apertures or two channels of the lower jaw, which design allows the suturing device to complete a “U” shaped stitch with a single pass. Thus, the suturing device as described herein can be suitable as a single-use, disposable device for surgeries where only one stitch is required, e.g., plantar plate surgery. In contrast, the apparatus described in Richardson's patents is designed to be re-loaded for multiple passes of the suture thread (see, e.g., col. 4, lines 59-62 of the '823 Patent).

Further, Richardson's design requires a portion of the suture thread to be relatively stiffened, e.g., by application of a stiffening agent such as epoxy resin. The suturing device as described herein does not require the suture thread to be used to undergo this extra manufacturing step.

Moreover, Richardson's mechanism for activating jaw elements differs from that of the suturing device described herein. Specifically, movement of the jaw elements according to Richardson's apparatus results from manipulating a pivotable actuation trigger, via levers, rather than rotation of an activation knob. Moreover, unlike the mechanism for activating jaw elements in Richardson's apparatus, the mechanism for activating jaw elements in the suturing device described herein is within the tubular body of the device, making it more simple and compact. Further, applicants are not aware of any suturing device which, like the suturing device described herein, can incorporate a separation segment in the drive shaft that, when the rotating knob is turned, shears the drive shaft allowing full jaw opening by the compressed spring.

U.S. Pat. No. 6,638,283 issued to Thal describes a suturing device having an actuation trigger similar to that disclosed in Richardson's patents. In Thal's device, the lower jaw is formed by a hollow tube having its end turned upwardly to face the upper jaw. In contrast, neither of the upper and lower jaws of the present invention is formed by a hollow tube having its end turned upwardly to face the upper jaw.

U.S. Pat. No. 6,770,084 issued to Bain et al. describes a lever actuated instrument that has a ramp on the jaws for directing the path of a needle. Unlike the device of the present invention, Bain's device does not comprise a pair of jaws, each of which comprises at least two separate apertures or channels configured and dimensioned to allow a suture thread to pass through.

U.S. Pat. No. 7,112,208 issued to Morris et al. describes a lever actuated instrument that pushes a malleable needle through a cannulation in a tube. U.S. Pat. Nos. 7,377,926 and 7,879,046 issued to Topper et al. describe instruments using bendable needles. The suturing device of the present invention does not utilize malleable or bendable needles. Moreover, the Morris patent and the Topper patents do not teach the rotatable actuation knob feature and the at least two apertures or channels in each jaw feature of the device as described herein.

### SUMMARY OF THE INVENTION

The present invention provides a suturing device comprising: (a) a lower jaw rigidly attached to an elongated suturing device body, said lower jaw comprises a needle retaining means for securing a pair of tissue perforating needles in position, and at least two apertures or channels within the lower jaw configured and dimensioned to allow a suture thread to pass through, and (b) an upper jaw mobile with respect to the lower jaw, said upper jaw comprises a needle capturing means for capturing the pair of tissue perforating needles, and at least two apertures or channels within the upper jaw configured and dimensioned to allow a suture thread to pass through, and (c) an activation mechanism for actuating movement of the upper jaw, wherein the needle retaining means and needle capturing means are configured such that when the upper jaw closes down on the lower jaw, and subsequently moves away from the lower jaw, the pair of tissue perforating needles is released by the lower jaw and simultaneously transferred to the upper jaw. The present invention also provides a suturing device comprising: (a) an elongated drive shaft comprising a first end and a second end housed in a protective tube, (b) a handle assembly comprising a tubular handle body, located on the first end of the drive shaft, (c) at least one movable element located on the second end of the drive shaft, which movable element is connected to the drive shaft, and (d) an activation mechanism for actuating movement of the at least one movable element, wherein: the activation mechanism comprises a rotatable actuation knob, movement of the drive shaft in its longitudinal direction is actuatable by rotation of the rotatable actuation knob about the longitudinal axis of the drive shaft, and movement of the movable element is actuated by movement of the drive shaft in its longitudinal direction. A suturing device combining the above-described jaw elements and activation mechanism for said jaw elements is additionally provided. Further, kits comprising disclosed devices and surgical methods utilizing the same are also provided.

### BRIEF DESCRIPTION OF THE DRAWINGS

FIG. 1 shows the exploded view of a suturing device according to an embodiment of the present invention.

FIG. 2 shows the side elevation view of a fully-assembled suturing device according to an embodiment of the present invention, having closed jaws.

FIG. 3 shows the side cross-sectional view of a fully-assembled suturing device according to an embodiment of the present invention, having open jaws.

FIGS. 4A-4C show the side cross-sectional view of a suturing device according to an embodiment of the present invention during the three steps of use. FIG. 4A shows step 1 of introducing the suturing device to the tissue (jaws open, needles in the lower jaw). FIG. 4B shows step 2 of capturing the tissue. FIG. 4C shows step 3 of removing the suturing device from the tissue (jaws open, needles in the upper jaw).

FIG. 5A shows a close-up view of detail BA (threaded section and spring within the handle body) of FIG. 4A. FIG. 5B shows a close-up view of detail AY (threaded section and spring within the handle body) of FIG. 4B. FIG. 5C shows a close-up view of detail BB (threaded section and spring within the handle body, and the separated drive shaft) of FIG. 4C.

FIGS. 6A-6F show the upper and lower jaws of the suturing device according to an embodiment of the present invention, where they have a toothed face. FIG. 6A shows the top elevation view of the lower jaw. FIG. 6B shows the side elevation view of the lower jaw. FIG. 6C shows a perspective view of the lower jaw. FIG. 6D shows the top elevation view of the upper jaw. FIG. 6E shows the side elevation view of the upper jaw. FIG. 6F shows a perspective view of the upper jaw.

FIGS. 7A and 7B show a first embodiment (FIG. 7A) and second embodiment (FIG. 7B) of a suturing thread according to the present invention having two tissue perforating needles connected thereto. FIG. 7C shows a close-up view of detail J (tissue perforating needles) of FIG. 7A.

FIGS. 8A-8C shows positioning of the suture thread within the suturing device. FIG. 8A shows the hollow space within the suturing device which can be used house the pre-loaded suture thread according an embodiment of the present invention. FIG. 8B shows a close-up view of the suture thread housed in a hollow space within the handle body according an embodiment of the present invention. FIG. 8C shows a close-up view of the suture thread having two tissue perforating needles attached at each end, which tissue perforating needles are positioned within the lower jaw of the suturing device according an embodiment of the present invention.

FIGS. 9A-9C show the perspective view of a suturing device according to an embodiment of the present invention during the three steps of use. FIG. 9A shows step 1 of introducing the suturing device to the tissue. In this step, the jaws are open for grasping tissue. FIG. 9B shows step 2 of capturing the tissue. In this step, jaws are closed, clamping the tissue, and perforating the tissue with needles. FIG. 9C shows step 3 of removing the suturing device from the tissue. In this step jaws are opened by drive shaft separation and spring driving the drive shaft forward. Needles are transferred by force fit into the upper jaw.

FIGS. 10A-10C show the close-up perspective views of the upper and lower jaws of the suturing device according to the embodiment shown in FIGS. 10A-10C. FIG. 10A shows step 1 of introducing the suturing device to the tissue. There is a loose fit between the lower jaw and the needles. FIG. 10B shows step 2 of capturing the tissue. It can be seen that the upper jaw aperture opening is smaller than that of the lower jaw so the needles can be captured by the upper jaw by force fit. FIG. 10C shows step 3 of removing the suturing device from the tissue.

### DESCRIPTION OF THE PREFERRED EMBODIMENT

Certain surgical procedures require manipulation of soft tissues which due to its location relative to the other bodily structures, e.g., joints, bones, internal organs, and tissue structures etc., preclude easy visualization and access. Often the targeted soft tissue is connected to hard tissues (bones). The original position of the targeted soft tissue may be less than ideal, resulting in impairment of movement of associated bone or joint structure. The purpose of the surgical procedure would then be to move the tissue connection point and anchoring it in a new location, so that the patient may return to normal activity and pain may be relieved. As an example, when joints of the foot are being treated, such as in plantar plate surgery, the soft tissue to be grasped can be underneath the bones of the toes and in between the soft tissue pad of the foot, neither of which should be moved or cut to gain access.

While aspects of the invention described herein are described with reference to plantar plate surgery, it should be appreciated that described device, kit and method can be used in other procedures requiring sutures, in particular those requiring single-stitch sutures.

### Definitions

As used herein, and unless stated otherwise, each of the following terms shall have the definition set forth below.

As used herein, “about” in the context of a numerical value or range means ±10% of the numerical value or range recited or claimed. By any range disclosed herein, it is meant that all hundredth, tenth and integer unit amounts within the range are specifically disclosed as part of the invention. Accordingly, “about” a recited value specifically includes that recited value. For example, a range of about 20° refers to all measurements within the range of ±10% of 20°, including 20°.

As used herein, an “aperture” in the upper jaw or lower jaw is an opening or hole through the jaw defined by a perimeter formed fully within the jaw. In contrast, as used herein a “channel” in the upper jaw or lower jaw is a slot or recess not defined by a perimeter formed fully within the jaw. For example, the channel can be a U or V shaped recess similar to that described in the Richardson patents.

As used herein, the terms “upper jaw” and “lower jaw” are used for ease of reference to mechanical elements of the invention in the description thereof. As elements of a hand-held device, whether one jaw is physically located above or below another can change depending on how the device itself is positioned and oriented. Therefore, the terms “upper jaw” and “lower jaw” are not intended to, and should not be construed to, mean that the upper jaw must always be physically located above the lower jaw regardless of the manner in which the device is being held by the user.

In a first embodiment of the present invention, a suturing device is provided comprising: (a) a lower jaw rigidly attached to an elongated suturing device body, said lower jaw comprises a needle retaining means for securing a pair of tissue perforating needles in position, and at least two apertures or channels within the lower jaw configured and dimensioned to allow a suture thread to pass through, and (b) an upper jaw mobile with respect to the lower jaw, said upper jaw comprises a needle capturing means for capturing the pair of tissue perforating needles, and at least two apertures or channels within the upper jaw configured and dimensioned to allow a suture thread to pass through, and (c) an activation mechanism for actuating movement of the upper jaw, wherein the needle retaining means and needle capturing means are configured such that when the upper jaw closes down on the lower jaw, and subsequently moves away from the lower jaw, the pair of tissue perforating needles is released by the lower jaw and simultaneously transferred to the upper jaw.

Although the description of the invention herein focuses on the first embodiment of the present invention, it is envisioned within the scope of the invention that the relative rigidity of the lower jaw and mobility of the upper jaw can be interchanged. That is, in a second embodiment of the present invention, a suturing device is provided comprising: (a) a lower jaw comprising a needle retaining means for securing a pair of tissue perforating needles in position, and at least two apertures or channels within the lower jaw configured and dimensioned to allow a suture thread to pass through, and (b) an upper jaw comprising a needle capturing means for capturing the pair of tissue perforating needles, and at least two apertures or channels within the upper jaw configured and dimensioned to allow a suture thread to pass through, and (c) an activation mechanism, wherein (i) the upper jaw is rigidly attached to an elongated suturing device body and the lower jaw is mobile with respect to the upper jaw, and (ii) the activation mechanism is for actuating movement of the lower jaw, and (iii) the needle retaining means and needle capturing means are configured such that when the lower jaw closes on the upper jaw, and subsequently moves away from the upper jaw, the pair of tissue perforating needles is released by the lower jaw and simultaneously transferred to the upper jaw. It should be understood that features of the first embodiment as described herein concerning relative movement of the pair of jaws would be suitably modified to accommodate the configuration according to the second embodiment.

In a third embodiment of the present invention, a suturing device is provided comprising: (a) an elongated drive shaft comprising a first end and a second end housed in a protective tube, (b) a handle assembly comprising a tubular handle body, located on the first end of the drive shaft, (c) at least one movable element located on the second end of the drive shaft, which movable element is connected to the drive shaft, and (d) an activation mechanism for actuating movement of the at least one movable element, wherein: the activation mechanism comprises a rotatable rotatable actuation knob, movement of the drive shaft in its longitudinal direction is actuatable by rotation of the rotatable actuation knob about the longitudinal axis of the drive shaft, and movement of the movable element is actuated by movement of the drive shaft in its longitudinal direction.

A suturing device combining the above-described jaw elements and activation mechanism for said jaw elements is additionally provided. In a particular a suturing device is provided comprising (a) an elongated drive shaft comprising a first end and a second end, housed in a protective tube, (b) a handle assembly comprising a tubular handle body, located on the first end of the drive shaft, (c) a lower jaw rigidly connected to the second end of the drive shaft, said lower jaw comprises a needle retaining means for securing a pair of tissue perforating needles in position, and at least two apertures or two channels within the lower jaw configured and dimensioned to allow a suture thread to pass through, (d) an upper jaw mobile with respect to the lower jaw, said upper jaw comprises a needle capturing means for capturing the pair of tissue perforating needles, and at least two apertures or two channels within the upper jaw configured and dimensioned to allow a suture thread to pass through, and (e) an activation mechanism for actuating movement of the upper jaw, wherein the needle retaining means and needle capturing means are configured such that when the upper jaw closes down on the lower jaw, and subsequently moves away from the lower jaw, the pair of tissue perforating needles is released by the lower jaw and simultaneously transferred to the upper jaw.

In one embodiment of the present invention, the activation mechanism comprises a mechanical actuator. In another embodiment, the mechanical actuator is a rotatable actuation knob. In a further embodiment, the activation mechanism actuates movement of the drive shaft along its longitudinal axis, which in turn actuates movement of the upper jaw.

In a preferred embodiment of a suturing device described herein, the drive shaft comprises a threaded section. In a further embodiment, the drive shaft further comprises a sacrificial section having a cross-section smaller than that of the threaded section, said sacrificial section preferably located adjacent to the threaded section. In yet another embodiment, the cross-sectional diameter of the sacrificial section is smallest along the length of the drive shaft.

Further optional features of a suturing device described herein include a spring mechanism, toothed face of the upper and the lower jaw, a means for limiting rotational range of the drive shaft about its longitudinal axis, and a means for limiting linear range of the drive shaft along its longitudinal axis. Further, the drive shaft can be connected to a male bore at the first end, and a mating female bore is located within the handle assembly or a component of the activation mechanism.

Also disclosed herein is a pre-assembled suture thread having tissue perforating needles at both ends to be fitted into the jaws of a suturing device, such as that described herein, so that as the soft tissue is grasped the tissue perforating needles penetrate the soft tissue and feed the suture thread through the perforations. The suture thread then can be grasped by another portion of the suturing device, another suturing device or another instrument, or manually by a member of the surgical staff, and can be secured in the preferred position or location. Further, kits comprising disclosed devices and surgical methods utilizing the same are also provided.

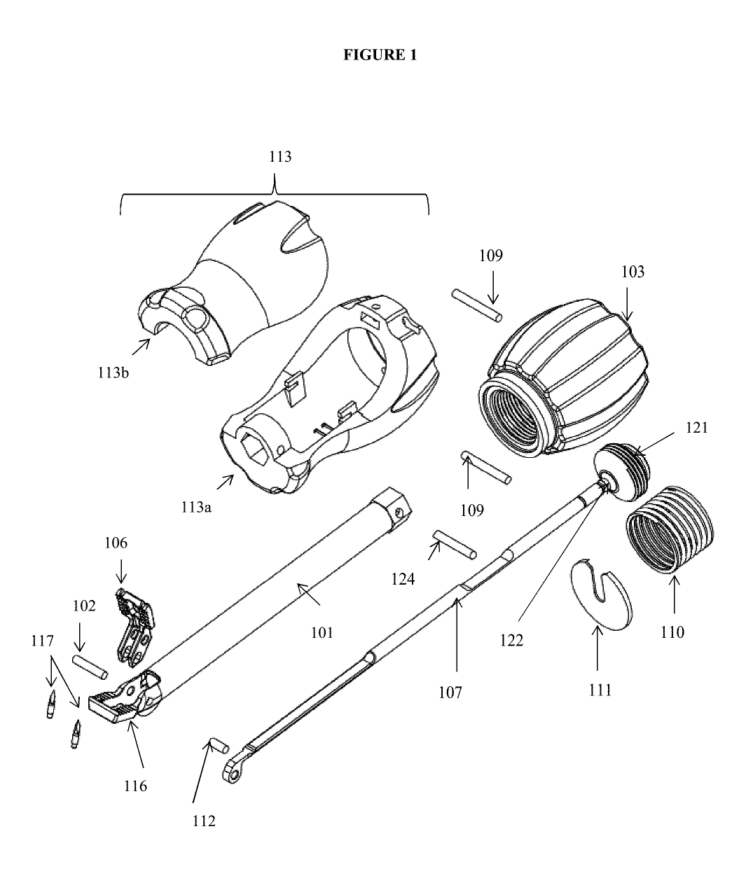

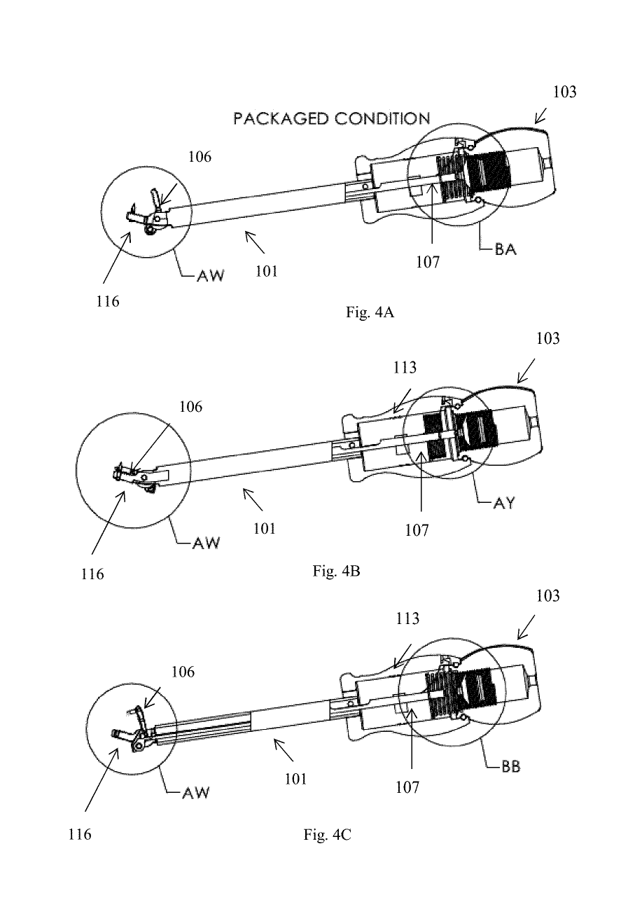

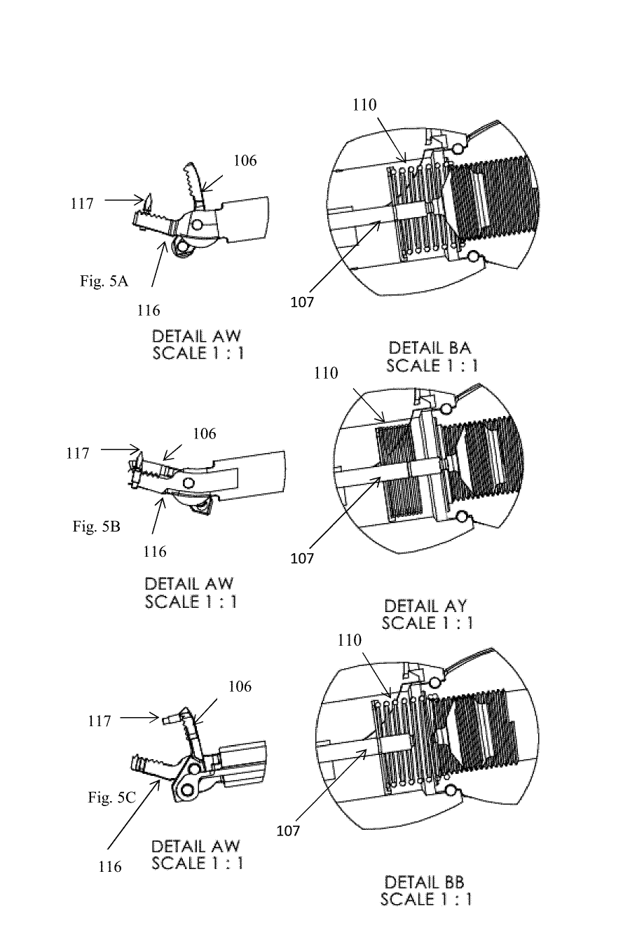

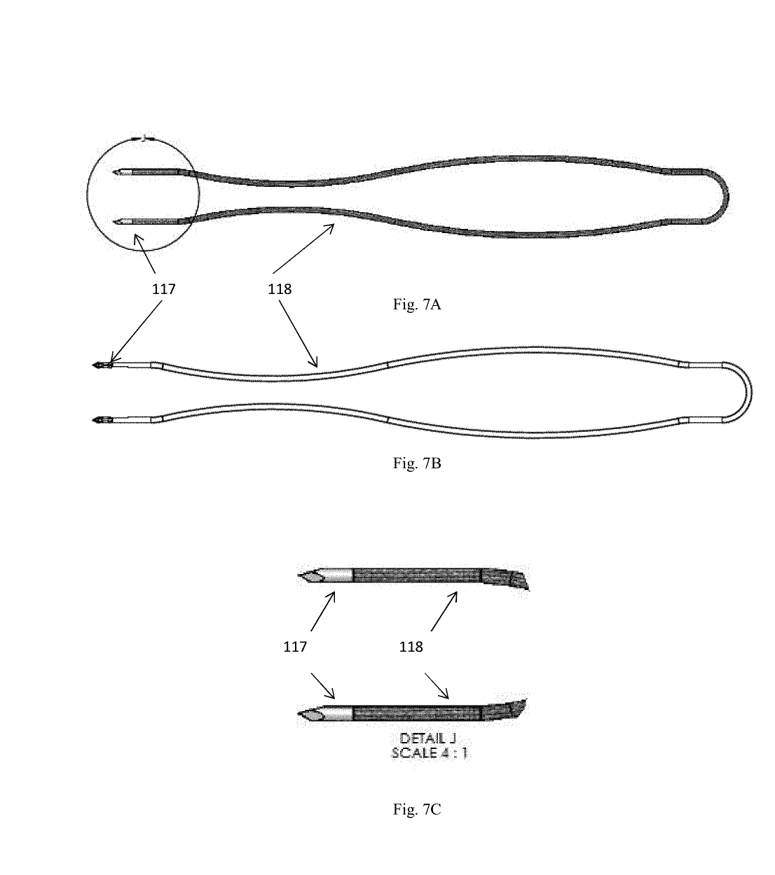

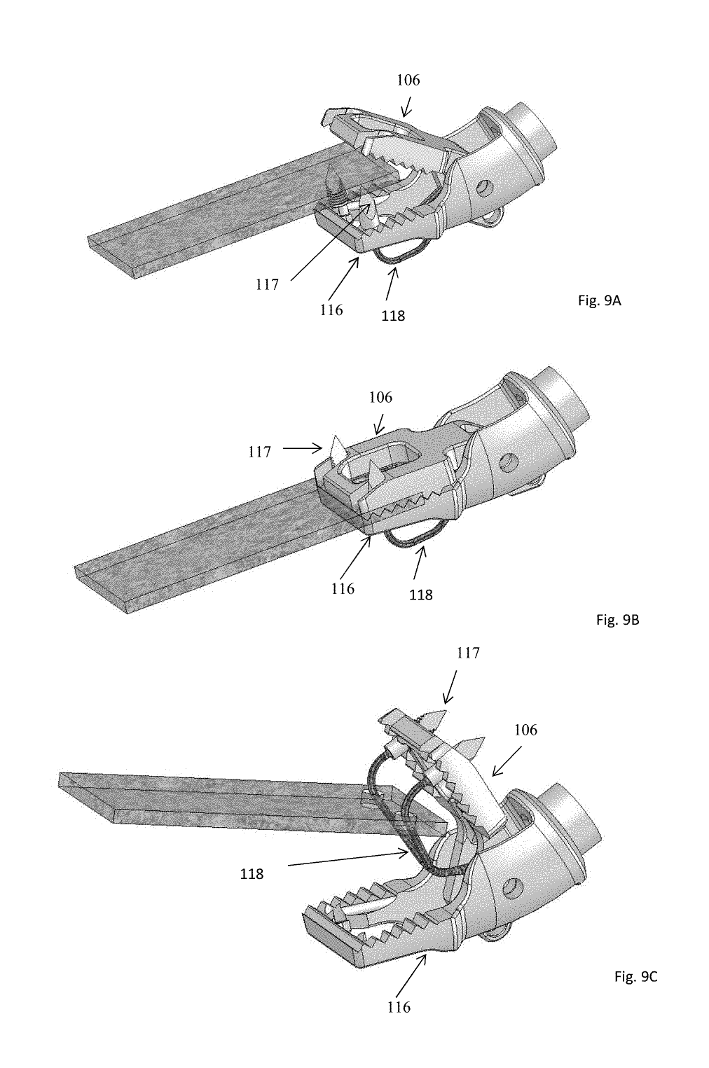

With reference to FIGS. 1-10, exemplary suturing devices and methods of using the same according to the present invention are described below. These examples are set forth to aid in understanding of the present invention but are not intended to, and should not be construed to, limit in any way the claims which follow thereafter. Further, in these figures, like or corresponding elements presented in different drawing figures are identified using the same reference numeral.

Specific embodiments and examples of the devices, kits and methods described herein are illustrative, and many variations can be introduced on these embodiments and examples without departing from the spirit of the disclosure or from the scope of the appended claims. Elements and/or features of different illustrative embodiments and/or examples may be combined with each other and/or substituted for each other within the scope of this disclosure and appended claims.

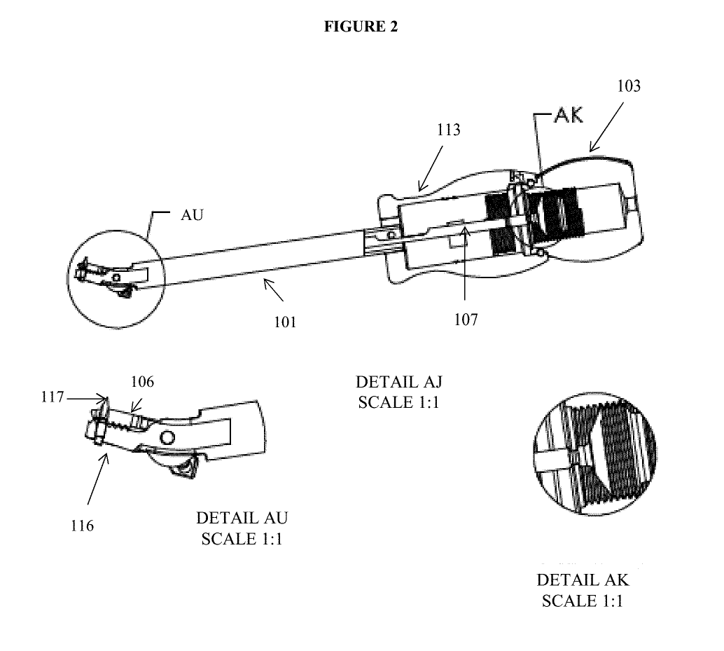

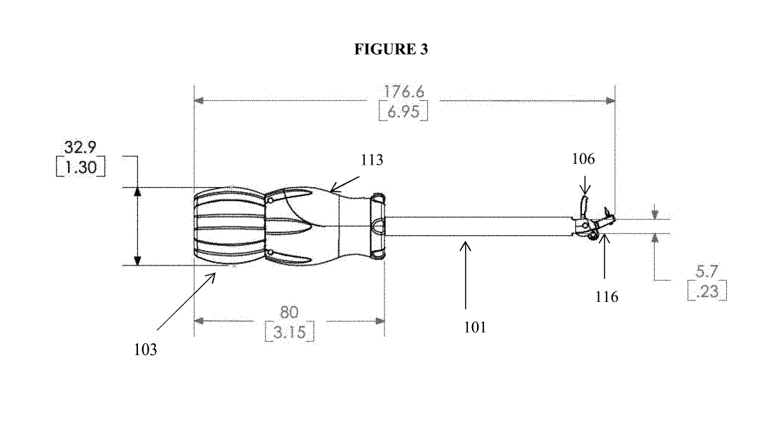

FIG. 1 shows an exploded view of a suturing device in accordance with an embodiment of the present invention comprising an outer protective tube (101), a jaw pin (102), an activation knob (103) having internal threads, an upper jaw (106), a drive shaft (107), a solid knob pin (109), a spring (110), a drive shaft washer (111), a drive shaft pin (112), a bottom solid handle (113a), a top solid handle (113b), a lower jaw (116), a tube pin (124), and two tissue perforating needles (117). Examples of fully-assembled suturing device according to the present invention can be seen in, e.g., FIGS. 2 and 3.

In use, the upper jaw (106) and the lower jaw (116) of the suturing device together grasp the targeted soft tissue and allow for its manipulation. The grasped tissue can be moved by pushing or pulling it into a desired location prior to being fastened in place to allow for a return to function.

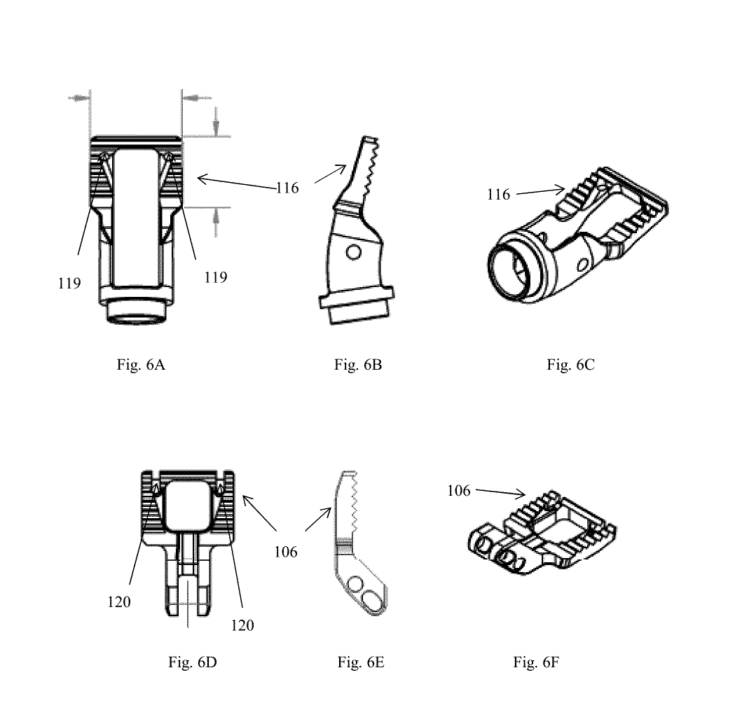

Close-up views of two embodiments of the upper jaw (106) and the lower jaw (116) can be found in FIGS. 6A-6F. The lower jaw (116) has two locations in the form of apertures or channels (119) that are shaped to retain the tissue perforating needles (117) while being positioned within the patient. As the movable upper jaw (106) is closed down upon the lower jaw (116) by a drive shaft (107), two complimentary apertures or channels (120) in the upper jaw (106) capture and rigidly hold the tissue perforating needles (117). The capturing of the tissue perforating needles (117) by the upper jaw (106) can be either by a force fit in the apertures or channels (120) of the upper jaw (106) or other means such as sacrificial materials that cause drag friction on tissue perforating needles (117) that are only loosely fitted into the lower jaw (116).

An important requirement of the above-described needle transfer feature is that the retention of the tissue perforating needles (117) in the lower jaw (116) by a needle retention means is minimal as compared to capturing force of the complimentary capturing element (the needle capturing means) found in the upper jaw (106). This requirement can be achieved in a variety of ways, for example, by appropriate sizing of interference fits, by deforming surfaces or by using a material that is displaced by the sharp tip of the tissue perforating needles (117). For example, the needles (117) can be initially only loosely fitted (free-running fit) in the lower jaw (116), and subsequently captured by a friction retention component in the upper jaw (106).

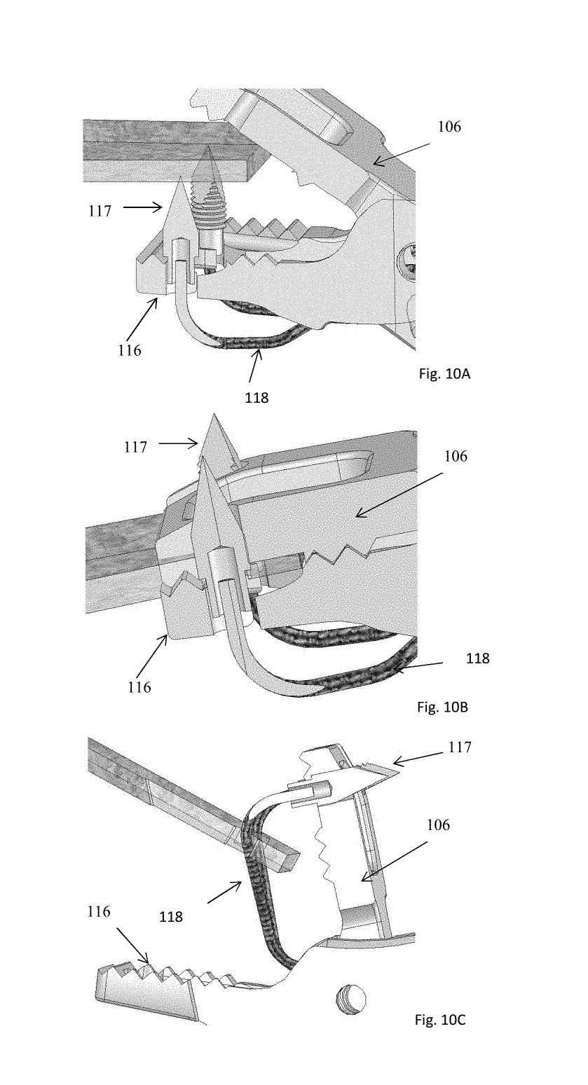

An example of the force-fit design can be seen in FIGS. 10A-10C. Specifically, as can be seen in FIGS. 10A-10C, there is a clearance between the size of the tissue perforating needles (117) and the sides of the apertures or channels (119) of the lower jaw (116). The fit of the needles (117) to the upper jaw (106) can be either a force fit of the outside diameter of the needle (117) to the jaw (106) or a sacrificial geometric surface shape, e.g. threads, knurls, on the outside diameter of the needle (117). The force fit can be formed by the deformation of either the body of the tissue perforating needles (117) or the upper jaw (106) or both.

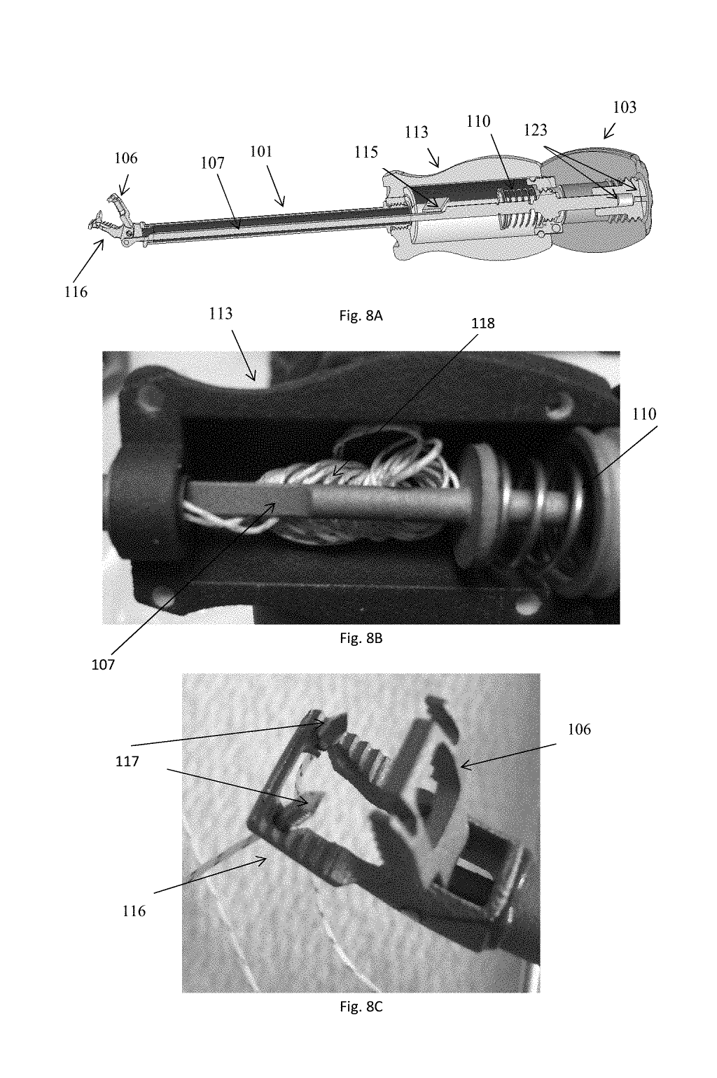

Both the upper and lower jaws have apertures or channels (119, 120) within them that allow for the passage of the suture thread (118) through them. This design serves to protect and guide the suture thread (118) as it is deployed within the patient and as it is pulled through the soft tissue outside of the suturing device. The jaws (106, 116) also have open passages, which can be seen in FIG. 8C, separate from the apertures or channels (119, 120), through the preferably toothed faces from top to bottom so that visualization of the jaws (106, 116) to tissue contact and grasped segment of soft tissue can occur. The opening leaves a perimeter of contact all the way around the jaw faces so that they can effectively grasp the sometimes delicate soft tissues requiring treatment. The teeth of the preferably toothed faces are preferably designed to mesh peak to valley so as to not cut into the soft tissues, but grasp the soft tissues effectively.

In comparison to the movable upper jaw (106), the lower jaw (116) is static and is rigidly connected to the handle body (113) of the suturing device by an outer protective tube (101). The upper jaw (106) is mobile and is activated by an elongated drive shaft (107) that is connected to the upper jaw (106) on one end and at the other end to an activation knob (103). When the drive shaft (107) is moved in a linear direction relative to the lower jaw (116), the upper jaw (106) closes and the preferably toothed faces of the jaws (106, 116) mesh together. This jaw closing also transfers the previously mentioned tissue perforating needles (117) from the lower jaw (116) to the upper jaw (106).

Of course, in the case of the second embodiment of the present invention, the upper would be static and rigidly connected to the handle body by the outer protective tube. The lower jaw would be mobile and activated by the elongated drive shaft connected to the lower jaw on one end and at the other end to the activation knob. When the drive shaft is moved in a linear direction relative to the upper jaw, the lower jaw would close and the preferably toothed faces of the jaws mesh together. This closing of the jaws transfers the tissue perforating needles from the lower jaw to the upper jaw.

The operation of the drive shaft (107) can be through a rotation of the activation knob (103) that drives a threaded section (121) of the drive shaft (107) in a linear direction along the axis of the instrument forward and backward within the outer protective tube (101). The outer protective tube (101) serves the purpose of both protecting other tissue structures within the patient and containing the suture thread (118) (see, e.g., FIG. 8A). The outer protective tube (101) is rigidly connected to the lower jaw (116) and the upper jaw (106) can be connected to the lower jaw (116) by a pin that allows the relative movement of the upper jaw (106) toward the lower jaw (116). Or, in the case of the second embodiment, the outer protective tube would be rigidly connected to the upper jaw and the lower jaw can be connected to the upper jaw by a pin that allows the relative movement of the lower jaw toward the upper jaw. The drive shaft (107) threaded section (121) at the opposite end is contained within a handle body (113) that is held in one hand while the activation knob (103) is turned by the physician.

Although the figures show actuation of the jaws (106, 116), via converting the rotary motion of the activation knob (103) into a linear motion of the drive shaft (107) can be accomplished using a male/female thread, alternative methods such as other types of mechanical linkages may be substituted. One example of such linkage is use of gear drives. Literatures in the relevant field, for example the Richardson patents, often teach use linear linkages in the form of moving handles. The handles redirect through levers the opening and closing of the handle to directly move the jaws open and close. These literature also, but often in separate mechanisms, teach moving the tissue perforating needles. A preferred embodiment of the device of the present invention combines both into one mechanism, making it more compact and simpler to operate. According to this design, the mechanical advantage required to perforate the tissue is generated by the sizing of the drive shaft to jaw linkage and the type and size of drive shaft threads (121) and the mating activation knob (3) threads.

The drive shaft (107) may comprise additional functional features. In an embodiment, a spring mechanism (110) is fixed to the drive shaft (107) that maintains tension on the drive shaft (107) and the jaws (106, 116) and with the next described feature serves to open the jaws (106, 116) after the needle perforation step. The drive shaft (107) can comprise a sacrificial section (122) which is an intentionally thinned cross-section at the end of the shaft before the threaded section (121). The threading feature converts the rotary motion of the activation knob (103) into the linear movement of the drive shaft (107) and through the pinned connection, the movement of the upper jaw (106). The length of engagement of the threaded connection between the drive shaft (107) and the activation knob (103) is specifically designed to move the drive shaft (107) only far enough to close the jaws (106, 116). When the distance of drive shaft movement is complete and the jaws (106, 116) are closed, an interference fit connection (123) at the end of the drive shaft (107) will impart a rotary motion and shear the drive shaft (107) at the sacrificial section (122) in front of the linear drive threads. In the accompanying figures, the interference fit connection (123) is a design having tapered bore that contacts the cylindrical end of the drive shaft (107). However, other mechanisms such as a geared face, or flat to flat could be used.

The sacrificial section of the drive shaft (107) is positioned to allow for the linear thread motion generating threaded feature to disengage when the jaws are closed. Specifically, the sacrificial section (122) shears due to rotary force twisting the thinned section of the drive shaft (107). In this manner, the sacrificial section (122) breaks when necessary so that further rotation of the activation knob (103) will no longer translate into linear motion of the drive shaft (107). Further, the force and motion necessary to break the sacrificial section (122) can be varied by varying the shape and amount of material in the cross section of the sacrificial section (122). The sizing of the male and female threaded connection is designed to allow for the jaws (106, 116) to close by linear movement of the drive shaft (107) and to no longer occur when the threads are no longer in contact with each other. U.S. Pat. No. 8,221,478 describes a snap-off surgical screw having similar features.

When the drive shaft (107) is separated, the previously mentioned spring (110) imparts a linear motion to the drive shaft (107) and the jaws (106, 116) are forced open. There may be a frictional interference fit (123) at the end of the drive shaft mechanism which is shown in, e.g., FIG. 2 or 8, as a simple male-female tubular connection. The female that contacts the end of the drive shaft (107) is a tube that has a tapered bore that, as the drive shaft (107) advances towards the female fitting surface contact, generates a force fit, and the continued rotation of the activation knob (103) is converted from a linear motion to a rotary motion. It should be noted that the drive shaft separation feature as above-described, i.e., having a tapered internal bore on a female part frictionally engaging the drive shaft end, is only one way of imparting a rotational load to shear the drive shaft. An alternative method is to use linkages or connections that engage like gears. Additionally, a flat can be machined into the end of the drive shaft (107), such that when the drive shaft (107) moves through the thread drive mechanism it comes into a flat in the activation knob (103) which at this point rotates and separates the shaft through the thinned shear area.

The primary purpose of the optional rotating shear feature is to render the suturing device no longer operational where the drive shaft (107) is separated from the linear thread drive feature, for when a single use device is desired. Of note, often times patients undergoing surgical procedures are suffering from pathologies which may or may not be related to the condition being treated, that reduce the rate of healing. It is beneficial to patients, in particular patients suffering from multiple conditions, to minimize the number of cuts and incisions made during surgical access.

The drive direction of the thread feature and the direction of the shearing function are in the same rotational direction so operation of the suturing device is as simple as possible for the physician. Further description of the sequence of events that occur during operation will follow. The handle body (113) has an internal hollow space sufficiently large to contain both the spring mechanism (110) on the drive shaft (107), the linear thread driving mechanism and the suture thread (118). As can be seen from FIG. 1, the handle body (113) can be designed so that it can be opened if needed. However, the instrument is preferably provided to the surgical staff preloaded and with the jaws (106, 116) in an open position, ready to be used. The handle body (113) preferably comprises feature(s) that serves to limit the rotation of the drive shaft (107) (see, e.g., FIG. 1 and FIG. 8A, drive guide plate (item 115)). An example of this feature is shown in the accompanying figures as a flat plate that is fitted to the handle body (113) and guides to movement of the jaw connecting end of the drive shaft (107). This plate also serves to resist the rotation of the lower end of the drive shaft (107) during the shaft shearing process. This part ensures that when the male and female shaft features begin to twist, the drive shaft (107) and the jaw connection are held and are not free to rotate due to the imparted torque, and rather focuses the torque loading on the sacrificial connection in the drive shaft (107).

The coil compression spring connected to the separable segment of the drive shaft can be another type of spring or can be attached in other areas on the drive shaft (107). Alternatively, the jaws (106, 116) themselves can have springs incorporated in them which serve to force the jaws open (106, 116) when capturing and penetration of the needles are completed.

### Method of Operation

The suturing device as described herein is preferably provided in a protective package that serves to maintain the cleanliness and sterility of the device, so that it is ready to use upon opening To use, the suturing device is preloaded with suture thread (118) having tissue perforating needles (117) on each end thereof. The physician grasps the loaded device with jaws (106, 116) at a preset open angle between them and inserts it in the area where the soft tissue segment is, then turns activation knob (103). The rotation of the activation knob (103) closes the jaws (106, 116) and as the jaws (106, 116) close the tissue perforating needles (117) that are assembled to the lower jaw (116) are transferred into the upper jaw (106). During the transfer, the tissue perforating needles (117) pass through the grasped soft tissue. Suture thread (118) that is assembled to the tissue perforating needles (117) base is also now positioned to be pulled through the perforated soft tissue to result in a “U” shaped suture. As the activation knob (103) is rotated the internal suture thread in the activation knob (103) and the external suture thread on the drive shaft (107) disconnect so no further movement of the jaws (106, 116) occur. At this point, the end of the drive shaft (107) comes into contact with the frictional interference (123), and the rotation of the activation knob (103) is converted from linear movement to rotary movement. The drive shaft (107) rotation is constrained by a part in the handle body (113) and the sacrificial section (122) shears. The captive spring moves the now loose drive shaft (107) segment forward opening the jaws (106, 116) completely and pulling the tissue perforating needles (117)/suture thread (118) assembly through the tissue and allows for the entire instrument to be removed from the surgical site. The suture thread (118) contained within the handle body (113) is allowed to be freely pulled out of the handle body (113) by the half cylindrical shape of the drive shaft (107) and the openings in the jaws (106, 116). The Surgeon can now use conventional practices and instruments to loop the single piece of suture thread (118) to “lasso” the soft tissue and attach it to other rigid structures in the patient. In a plantar plate procedure, this is typically completed by anchoring it to the distal toe bone from the one being sectioned. The anchoring procedure can be accomplished using a specially-designed drill that has a slot and an opening to allow for easy drilling the bone, then hooking the suture thread (118) which is then pulled back through the drill hole and tied to secure. The suture can be closed by cutting off the tip of the suture thread (118) containing the tissue perforating needles (117), which are, e.g., still frictionally fitted to the upper jaw (106) of the suturing device, then tying knots according to conventional surgical practices.

Finally, the combination of any embodiment or feature mentioned herein with one or more of any of the other separately mentioned embodiments or features is contemplated to be within the scope of the instant invention.

1. A suturing device comprising:
(a) an elongated drive shaft comprising a first end and a second end, housed in a protective tube,
(b) a handle assembly comprising a tubular handle body, located on the first end of the drive shaft,
(c) a lower jaw rigidly connected to the second end of the drive shaft, said lower jaw comprises a needle retaining means for securing a pair of tissue perforating needles in position, and at least two apertures or two channels within the lower jaw configured and dimensioned to allow a suture thread to pass through,
(d) an upper jaw mobile with respect to the lower jaw, said upper jaw comprises a needle capturing means for capturing the pair of tissue perforating needles, and at least two apertures or two channels within the upper jaw configured and dimensioned to allow a suture thread to pass through, and
(e) an activation mechanism for actuating movement of the upper jaw,
wherein the needle retaining means and needle capturing means are configured such that when the upper jaw closes down on the lower jaw, and subsequently moves away from the lower jaw, the pair of tissue perforating needles is released by the lower jaw and simultaneously transferred to the upper jaw.

2. The suturing device of claim 1, wherein the activation mechanism comprises a mechanical actuator.

3. The suturing device of claim 2, wherein the mechanical actuator is a rotatable actuation knob.

4. The suturing device of claim 1, wherein the activation mechanism actuates movement of the drive shaft along its longitudinal axis, which in turn actuates movement of the upper jaw.

5. The suturing device of claim 1, wherein the drive shaft comprises a threaded section.

6. The suturing device of claim 5, wherein the drive shaft further comprises a sacrificial section having a cross-section smaller than that of the threaded section, said sacrificial section preferably located adjacent to the threaded section.

7. The suturing device of claim 6, wherein cross-sectional diameter of the sacrificial section is smallest along the length of the drive shaft.

8. The suturing device of claim 1, further comprising a spring mechanism.

9. The suturing device of claim 1, wherein the drive shaft is connected to a male bore at the first end, and a mating female bore is located within the handle assembly or a component of the activation mechanism.

10. The suturing device of claim 1, wherein a face of the upper jaw facing the lower jaw and a face of the lower jaw facing the upper jaw, are toothed.

11. The suturing device of claim 1, further comprising one or both of a means for limiting rotational range of the drive shaft about its longitudinal axis and a means for limiting linear range of the drive shaft along its longitudinal axis.

12. A suturing device comprising:
(a) a lower jaw rigidly attached to an elongated suturing device body, said lower jaw comprises a needle retaining means for securing a pair of tissue perforating needles in position, and at least two apertures or channels within the lower jaw configured and dimensioned to allow a suture thread to pass through, and
(b) an upper jaw mobile with respect to the lower jaw, said upper jaw comprises a needle capturing means for capturing the pair of tissue perforating needles, and at least two apertures or channels within the upper jaw configured and dimensioned to allow a suture thread to pass through,
(c) an activation mechanism for actuating movement of the upper jaw,
wherein the needle retaining means and needle capturing means are configured such that when the upper jaw closes down on the lower jaw, and subsequently moves away from the lower jaw, the pair of tissue perforating needles is released by the lower jaw and simultaneously transferred to the upper jaw.

13. A suturing device comprising:
(a) an elongated drive shaft comprising a first end and a second end housed in a protective tube,
(b) a handle assembly comprising a tubular handle body, located on the first end of the drive shaft,
(c) at least one movable element located on the second end of the drive shaft, which movable element is connected to the drive shaft,
(d) an activation mechanism for actuating movement of the at least one movable element,
wherein:
the activation mechanism comprises a rotatable actuation knob,
movement of the drive shaft in its longitudinal direction is actuatable by rotation of the rotatable actuation knob about the longitudinal axis of the drive shaft, and
movement of the movable element is actuated by movement of the drive shaft in its longitudinal direction.

14. A kit comprising:
a) the suturing device of claim 1; and
b) a suture thread having two ends, and a tissue perforating needles at each end.

15. A method for suturing a soft tissue comprising the steps of
a) providing a suture thread having two ends, and a tissue perforating needles at each end;
b) perforating the soft tissue by passing the tissue perforating needles through the soft tissue simultaneously; and
c) pulling the tissue perforating needles away from the perforated tissue, thereby pulling the suture thread through the soft tissue.
